(19) United States
(12) Patent Application Publication
(10) Pub. No.: US 2010/0274198 A1
(43) Pub. Date: Oct. 28, 2010

(54) INJECTION DEVICE

(75) Inventor: Herbert Bechtold, VS-Schwenningen (DE)

Correspondence Address: Oliver Intellectual Property LLC, P.O. Box 1670, Cotuit, MA, 02635, US

(73) Assignee: HASELMELER GMBH, St. Gallen (CH)

(21) Appl. No.: 12/740,097

(22) PCT Filed: Jul. 15, 2009
(86) PCT No.: PCT/EP2009/005123

§ 371 Date: Apr. 27, 2010

(30) Foreign Application Priority Data
Aug. 18, 2008 (DE) 20 2008 011 175.7

(51) Int. Cl. A61M 5/31

(52) U.S. Cl. 604/189

(57) ABSTRACT

An injection device has a housing (38) having a housing wall and having a first window (50) provided therein. Arranged rotatably in the housing (38) is a graduation carrier (60) on which are arranged, in helical fashion, dose values (62′) that are at least partly visible through the first window (50). A second window (54) is located in the housing (38) and is arranged displaceably in the longitudinal direction of the housing (38), a rotary motion of the graduation carrier (60) being synchronized with a longitudinal displacement of the second window (54) in order to indicate, through the first window (50) and the second window (54), the injection dose value (62′) that is presently selected. Since the selected dose value (62′) is only legible when viewed through both lenses (80,90), this militates against incorrect readings of the selected dose setting.

### CROSS-REFERENCE TO RELATED APPLICATION(S)

The present application is a section 371 of PCT/EP09/05123, filed 15 Jul. 2009, which in turn claims priority from German application DE 20 2008 011 175.7, filed 18 Aug. 2008, the disclosure of which is hereby incorporated by reference.

### FIELD OF THE INVENTION

The invention relates to an injection device that is equipped with an apparatus for indicating an injection dose that has been set.

### BACKGROUND

Ambiguous indications must not occur with such injection devices, since injection devices are often used by persons with poor vision, for example by diabetics, for whom an unequivocal display is extremely important, in order to avoid improper operation.

### SUMMARY OF THE INVENTION

It is therefore an object of the invention to make available a novel injection device which militates against improper operation.

According to the invention, this object is achieved by equipping the injection device with a housing having a first window therein, a graduation carrier bearing dose values arranged on its surface in a helical configuration, a longitudinally displaceable second window in the housing, and respective lenses in the first and second windows, such that the first lens by itself distorts the dose values but the first and second lenses interact to render a single dose value legible, thereby militating against incorrect readings of the dose value selected by the user. Only at the point, where the first transparent lens arrangement and the second transparent lens arrangement interact, is it possible for a readily legible display, of the dose that has been set, to be visible; whereas at points where only the first transparent lens arrangement, but not the second, is effective, the visible, but optically distorted, dose values do not permit a legible reading, and thereby militate against improper settings of the injection dose.

### BRIEF FIGURE DESCRIPTION

Further details and advantageous refinements of the invention are evident from the exemplifying embodiment, in no way to be understood as a limitation of the invention, that is described below and depicted in the drawings.

FIG. 1 shows an example of an injection device in which the invention can be utilized;

FIG. 2 shows the injection device of FIG. 1, on the left after an injection, on the right after an injection dose of 40 units has been set and prior to an injection; for a better comparison, the effect of the lens arrangement described here is depicted only in FIG. 2b, but not in FIG. 2a;

FIG. 3 presents three depictions of a housing part 38, used in the context of FIGS. 1 and 2, having a first window 50 that has a function in the context of indication of the injection dose that has been set; FIG. 3a is a plan view looking in the direction of an arrow IIIa of FIG. 3b; FIG. 3b is a section viewed along line IIIb-IIIb of FIG. 3a; FIG. 3c is a plan view of the window side looking in the direction of an arrow IIIc of FIG. 3b;

FIG. 4 is a perspective depiction of an inner sleeve used in the context of FIGS. 1 and 2, having a second window that likewise has a function in the context of indication of the injection dose that has been set;

FIG. 5 depicts the setting sleeve used in the context of FIGS. 1 and 2, which carries indicia that serve to indicate the injection dose that has been set;

FIG. 6 depicts the upper part of the injection device of FIG. 1;

FIG. 7 is a plan view of a first dispersing panel that, according to FIG. 6, is inserted into the first window of housing 38, viewed in the direction of arrow VII of FIG. 8;

FIG. 8 is a section viewed along line VIII-VIII of FIG. 7;

FIG. 9 is a plan view of a second dispersing panel that is inserted into second window 54 and interacts with the first dispersing panel;

FIG. 10 is a section viewed along line X-X of FIG. 9;

FIG. 11 is a depiction analogous to FIG. 6; and

FIG. 12 is a section viewed along line XII-XII of FIG. 11.

### DETAILED DESCRIPTION

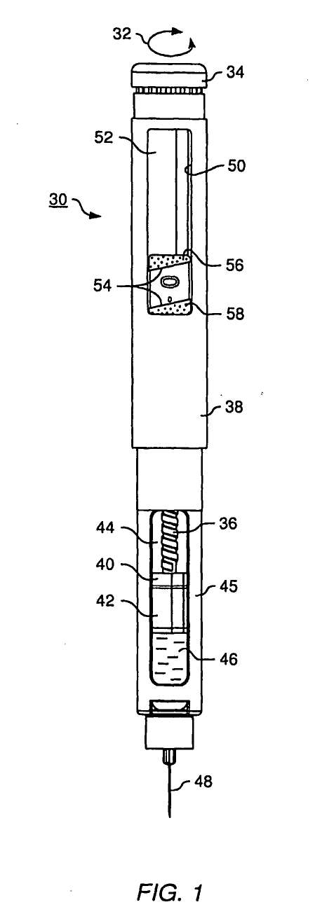

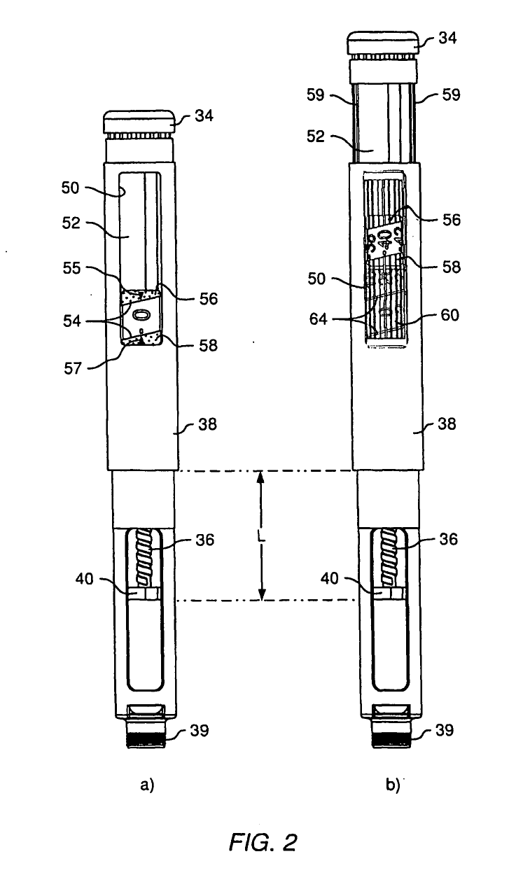

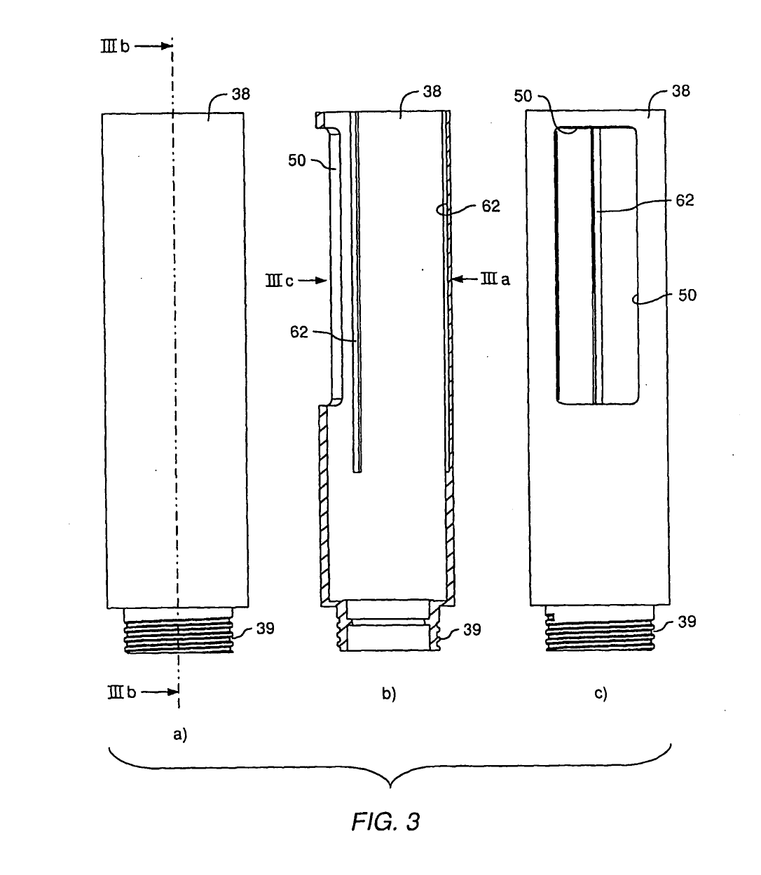

FIG. 1 is a side view of an injection device 30. This example refers to a so-called semiautomatic unit, i.e. the patient sets the desired injection dose by rotation 32 of a setting knob 34, as shown by a comparison of FIG. 2a (before setting) and FIG. 2b (after setting). A length L, by which a piston rod 36 projects out of a housing part 38, does not change in this context. Piston rod 36 has, at its lower end, an enlargement 40 with which it abuts against a rubber piston 42 that is arranged displaceably in a glass cartridge 44 in which injection fluid 46 is present. Glass cartridge 44 is located in a lower (proximal) housing part 45 that is screwed by means of a thread 39 (FIG. 3) onto upper housing part 38. An injection needle 48 (FIG. 1) is mounted on the lower portion of housing part 45 prior to an injection.

The terms “proximal” and “distal” are used in the manner that is usual in medicine, i.e. proximal=close to the patient (the end having needle 48), distal=away from the patient (the end having setting knob 34).

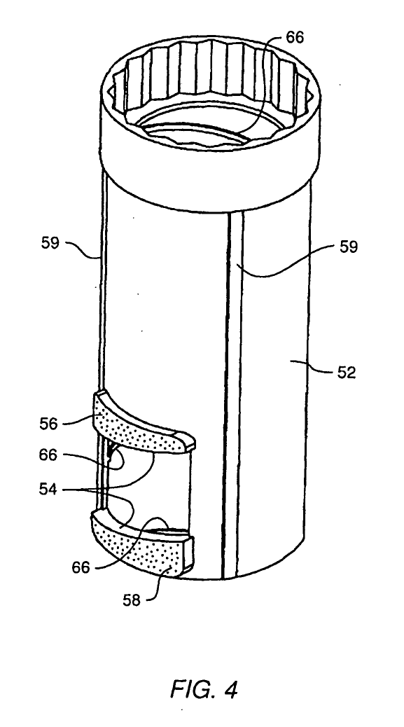

Upper housing part 38 has a first window 50 (FIG. 1) for setting an injection dose. A cylindrical inner sleeve 52 (FIG. 4) is arranged axially displaceable in upper housing part 38. Said sleeve has a second window 54 having an upper axial delimiter 56 away from the patient, and a lower axial delimiter 58 close to the patient. Inner sleeve 52 further has, on its outer side, axially extending ribs 59 that serve for axial guidance in corresponding inner grooves 62 (FIG. 3) of upper housing part 38.

Upper delimiter 56 acts as a stop for the maximum dose, which here is equal to approximately 60 units; and lower delimiter 58 acts as a stop for the zero dose, which is reached automatically after an injection. (This is depicted in FIG. 1.) Any intermediate positions are possible between these extreme values, i.e. 0 units and 60 units. A dose that was originally set and was too high can also be corrected by the patient.

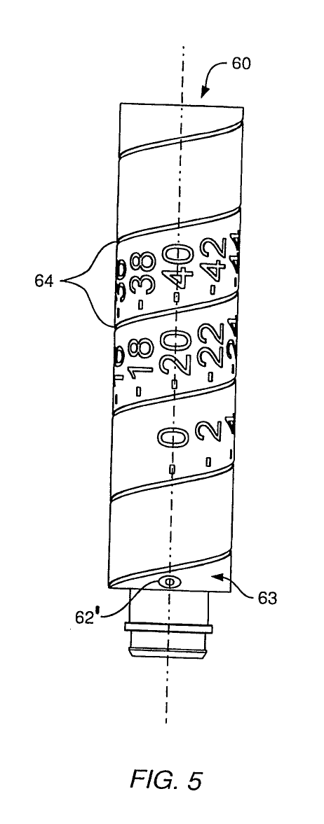

FIG. 5 shows graduated tube 60 on which is located scale 62′ having values 63 settable by the patient. These are arranged helically on the outer side of graduated tube 60. The latter is not axially displaceable relative to upper housing 38 but is rotatable, so that by means of setting knob 34 and a coupling (not depicted), it can be rotated relative to upper housing part 38. Graduated tube 60 has, on its outer side, an external thread 64 that is in engagement with an internal thread 66 of inner sleeve 52.

When graduated tube 60 is rotated by means of setting knob 34, it therefore brings about, using the two threads 64, 66, an axial displacement of inner sleeve 52 and thus of second window 54, specifically synchronously with the rotation of graduated tube 60, so that each dose indication of graduated tube 60 corresponds to a specific axial position of second window 64 relative to first window 50. An unequivocal dose indication thus results with this embodiment, since only the dose quantity, that is set, can be read in window 54.

If threaded sleeve 52 is short for design-related reasons, it may cover not the entire graduated tube 60 but only its upper part, as depicted in FIG. 1. The lower part of graduated tube 60, however, remains visible as depicted in FIG. 2b; this might confuse some patients.

This is because, in certain cases, the overall length of injector 30 must be minimized, so that the length of inner sleeve 52 (having window 54) cannot cover scale 62′ on its proximal region close to the patient. If this were the case, then in fact only the dose that has been set would be visible in window 54. For space reasons, however, this is not possible (see FIG. 2b).

This problem is solved by using housing part 38 having said first window 50, in which inner sleeve 52 having second window 54 is arranged in longitudinally displaceable fashion. Window 54 has an upper delimiter 56 and a lower delimiter 58. Indicator arrows 55, 57 that indicate the injection dose that has been set (see FIG. 2a) can be located on both delimiters.

Because the proximal part of scale 62′ on graduated tube 60 can no longer, because of the minimized length of inner sleeve 52, be completely covered upon axial displacement of inner sleeve 52 in a distal direction, not only the dose that has been set, but instead all the numbers of scale 62′, would be visible in the proximal region, close to the patient, of first window 50, since they are no longer concealed by inner sleeve 52.

For patients with poor vision, for example diabetics, this leads to the risk of misreading, i.e. in the case of, for example, the dose setting in accordance with FIG. 2b, a patient with poor vision might read off not the dose of 40 units that is set, but the value of 20 units or a value of 0, which might cause him or her to set an incorrect injection dose.

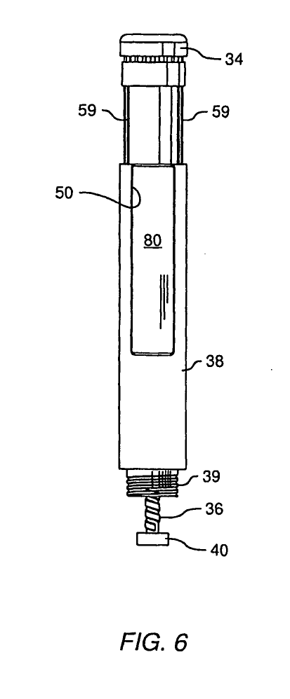

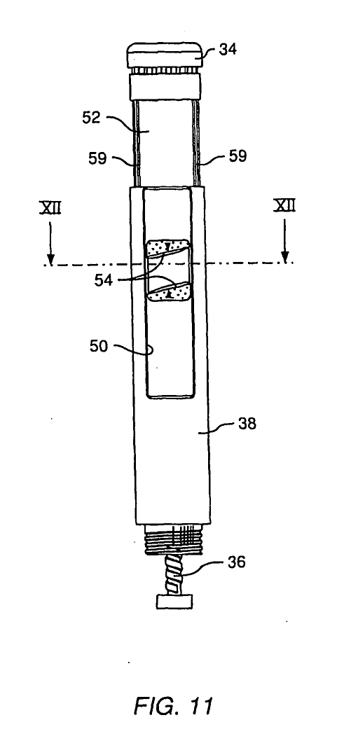

Such problems can be avoided with the configuration according to FIGS. 6 to 12.

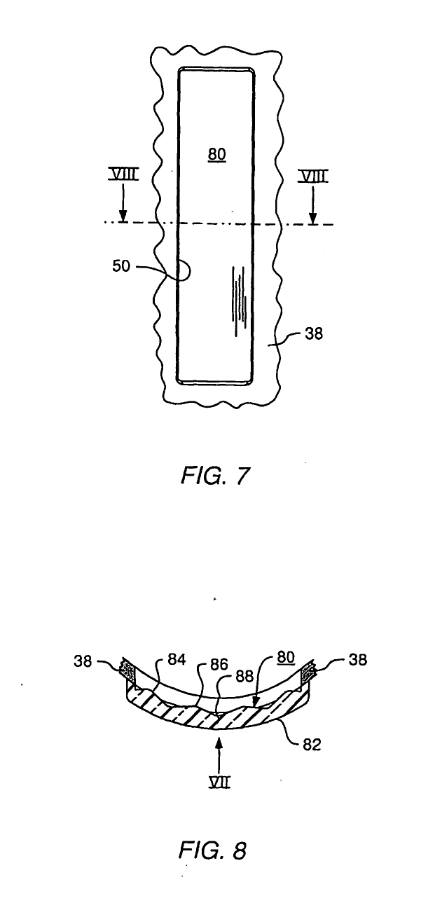

Here a first lens system 80, depicted in FIG. 7 and FIG. 8, is inserted into first window 50. Said system has the properties of a dispersing panel, i.e. prevents the patient from making incorrect readings through said first lens system 80.

As FIGS. 7 and 8 show, here the first transparent lens arrangement 80 is inserted into window 50, for example, by being clipped or adhesively bonded.

As FIG. 8 shows, lens 80 has in cross section a profile that is smooth on upper side 82, e.g. in the shape of a cylindrical surface or even a flat surface (not depicted). On lower side 84, facing away from upper side 82, lens 80 has a profile with elevations 86 and depressions 88 proceeding in a longitudinal direction. As a result, when lens 80 is used alone it acts as a dispersing panel, i.e. it is not possible to legibly read numbers 62′ of graduated tube 60 through said lens 80.

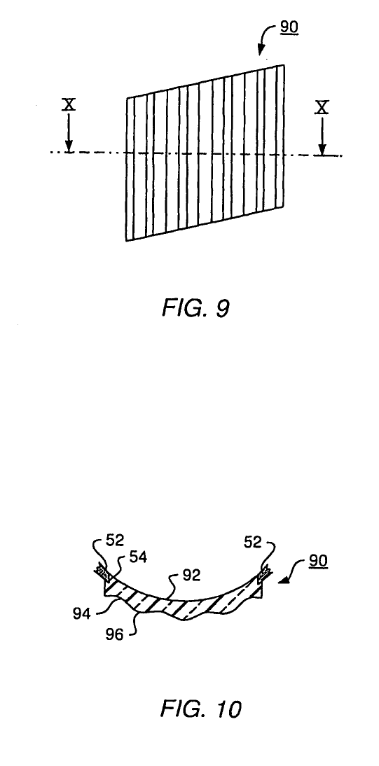

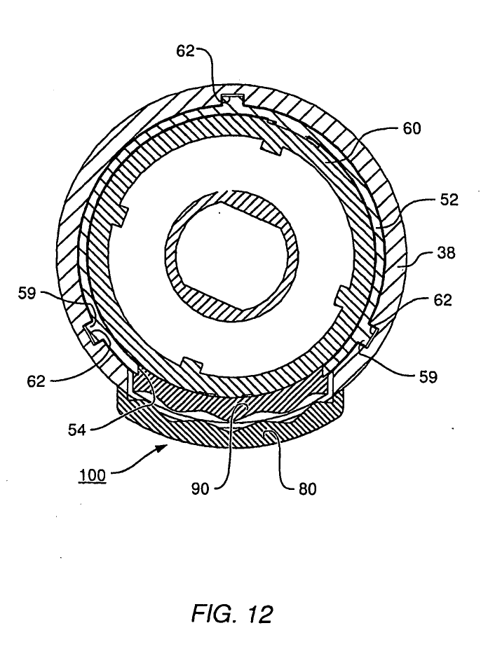

FIG. 9 and FIG. 10 show a corresponding configuration of a second transparent lens arrangement 90 that is implemented to interact with lens arrangement 80 (see FIG. 12).

Arrangement 90 is inserted into window 54 of inner sleeve 52, for example by being clipped or adhesively bonded in. Its lower side 92 is cylindrical or flat (not depicted), and its upper side has valleys 94 extending in a longitudinal direction and longitudinal ridges 96, which extend in complementary fashion to elevations 86 and depressions 88 of FIG. 8, so that first lens arrangement 80 and second lens arrangement 90, when they are arranged one above another with a spacing as depicted in FIG. 12, together form a lens arrangement 100.

Second lens arrangement 90 is displaceable in a longitudinal direction relative to first lens arrangement 80, so that the visible region within second window 54 migrates axially when graduated tube 60 is rotated for dose setting purposes. In this manner, only the dose value that is presently set can be read clearly, as depicted by way of example in FIG. 2b but not in FIGS. 1 and 2a.

(Be it noted, in this connection, that this optical effect is difficult to depict graphically.)

This improved legibility is achieved by the interaction of lens arrangement 100 with a mechanical setting apparatus. Incorrect settings are thereby reliably avoided, resulting in a readily understandable mode of operation.

Numerous variants and modifications are of course possible within the scope of the present invention. For example, inner sleeve 52 can likewise be made of transparent plastic, and can be formed integrally with second lens arrangement 90. It is particularly advantageous that, in the version according to FIGS. 7 to 12, inner sleeve 52 can be short (see FIG. 4).

Numerous variants and modifications are of course possible, within the scope of the present invention.

What is claimed is:

1. An injection device comprising:
a housing (38) having a housing wall and having a first window (50) provided therein;
a graduation carrier (60) arranged rotatably in the housing (38), on which carrier are arranged, in helical fashion, dose values (62′) that are at least partly visible through the first window (50);
a longitudinally displaceable second window (54) arranged in the housing (38), which window is arranged displaceably in the longitudinal direction of the housing (38), a rotary motion of the graduation carrier (60) being synchronized with a longitudinal displacement of the second window (54) in order to indicate, through the first window (50) and the second window (54), the injection dose value (62′) that is presently set;
a first transparent lens arrangement (80) arranged in the first window (50), which arrangement by itself brings about a distortion of the indicated values (62′) visible through the first lens arrangement (80); and
a second transparent lens arrangement (90) arranged in the second window (54), which arrangement, in interaction with the first transparent lens arrangement (80), increases the legibility of a dose value (62′) that has been set and is visible through both the first transparent lens arrangement (80) and the second transparent lens arrangement (90), and thereby militates against incorrect readings of the selected dose setting.

2. The injection device according to claim 1, wherein
the first transparent lens arrangement (80) is equipped, on its inner side (84) facing toward the second window (54), with elevations (86) and depressions (88) that extend in the longitudinal direction of said lens arrangement (80).

3. The injection device according to claim 2, wherein
the second transparent lens arrangement (90) is equipped, on its outer side facing toward the first transparent lens arrangement (80), with depressions (94) and elevations (96) which have a profile that is adapted to the elevations (86) and depressions (88) of the first transparent lens arrangement (80).

4. The injection device according to claim 1, wherein
the first transparent lens arrangement (80) is shaped on its outer side (82) as a cylindrical segment.

5. The injection device according to claim 1, wherein
the second transparent lens arrangement (90) is formed on its inner side (92) as a hollow cylindrical segment.

6. The injection device according to claim 1, wherein
a bearing arrangement is provided for rotatable journaling of the graduation carrier (60) relative to the housing (38).

7. The injection device according to claim 6, wherein
the bearing arrangement is implemented so that it counteracts an axial displacement between the housing (38) and graduation carrier (60).

8. The injection device according to claim 1, wherein
an external thread (64) is provided on the outer side of the graduation carrier (60), which thread is in engagement with an internal thread (66) that is so configured, adjacent the second window (54), that a rotation of the graduation carrier (60) through a predefined rotation angle corresponds to a predefined longitudinal displacement of the second window (54).

9. The injection device according to claim 2, wherein
the first transparent lens arrangement (80) is shaped on its outer side (82) as a cylindrical segment.

10. The injection device according to claim 3, wherein
the first transparent lens arrangement (80) is shaped on its outer side (82) as a cylindrical segment.

11. The injection device according to claim 4, wherein
the second transparent lens arrangement (90) is formed on its inner side (92) as a hollow cylindrical segment.

12. The injection device according to claim 9, wherein
the second transparent lens arrangement (90) is formed on its inner side (92) as a hollow cylindrical segment.

13. The injection device according to claim 10, wherein
the second transparent lens arrangement (90) is formed on its inner side (92) as a hollow cylindrical segment.

14. The injection device according to claim 2, wherein
a bearing arrangement is provided for rotatable journaling of the graduation carrier (60) relative to the housing (38).

15. The injection device according to claim 3, wherein
a bearing arrangement is provided for rotatable journaling of the graduation carrier (60) relative to the housing (38).

16. The injection device according to claim 2, wherein
an external thread (64) is provided on the outer side of the graduation carrier (60), which thread is in engagement with an internal thread (66) that is so configured, adjacent the second window (54), that a rotation of the graduation carrier (60) through a predefined rotation angle corresponds to a predefined longitudinal displacement of the second window (54).

17. The injection device according to claim 3, wherein
an external thread (64) is provided on the outer side of the graduation carrier (60), which thread is in engagement with an internal thread (66) that is so configured, adjacent the second window (54), that a rotation of the graduation carrier (60) through a predefined rotation angle corresponds to a predefined longitudinal displacement of the second window (54).

18. The injection device according to claim 4, wherein
an external thread (64) is provided on the outer side of the graduation carrier (60), which thread is in engagement with an internal thread (66) that is so configured, adjacent the second window (54), that a rotation of the graduation carrier (60) through a predefined rotation angle corresponds to a predefined longitudinal displacement of the second window (54).

19. The injection device according to claim 5, wherein
an external thread (64) is provided on the outer side of the graduation carrier (60), which thread is in engagement with an internal thread (66) that is so configured, adjacent the second window (54), that a rotation of the graduation carrier (60) through a predefined rotation angle corresponds to a predefined longitudinal displacement of the second window (54).
